(19) United States
(12) Patent Application Publication
(10) Pub. No.: US 2004/0023265 A1
(43) Pub. Date: Feb. 5, 2004

(54) Methods and compositions for nucleic acid ligands against Shiga toxin and/or Shiga-like toxin

(76) Inventors: Jeevalatha Vivekananda, San Antonio, TX (US); Johnathan L. Kiel, Universal City, TX (US)

Correspondence Address: Blakely Sokoloff Taylor & Zafman, Seventh Floor, 12400 Wilshire Boulevard, Los Angeles, CA, 90025-1030, US

(21) Appl. No.: 10/386,778

(22) Filed: Mar. 11, 2003

Related U.S. Application Data
(63) Continuation-in-part of application No. 09/978,753, filed on Oct. 15, 2001, now Pat. No. 6,569,630.
Application No. 09/978,753 is a continuation-in-part of application No. 09/909,492, filed on Jul. 19, 2001, now abandoned.
Application No. 09/909,492 is a continuation-in-part of application No. 09/608,706, filed on Jun. 30, 2000, now Pat. No. 6,303,316.
(60) Provisional application No. 60/379,904, filed on May 10, 2002.
(60) Provisional application No. 60/142,301, filed on Jul. 2, 1999.
(60) Provisional application No. 60/199,620, filed on Apr. 25, 2000.

(51) Int. Cl. C12Q 1/68

(52) U.S. Cl. 435/6

(57) ABSTRACT

The present invention concerns methods of preparing nucleic acid ligands against Shiga toxin and/or Shiga-like toxin, compositions comprising nucleic acid ligands that bind Shiga toxin and/or Shiga-like toxin, nucleic acid ligands comprising contiguous nucleotide sequences selected from SEQ ID NO:1 through SEQ ID NO:11 and methods of use of such ligands for detection and/or neutralization of Shiga toxin and/or Shiga-like toxin.

### RELATED APPLICATIONS

The present application claims the benefit under 35 U.S.C. §119(e) of U.S. Provisional Patent Application Serial No. 60/379,904, filed May 10, 2002. This application is a continuation-in-part of U.S. patent application Ser. No. 09/978,753, filed Oct. 15, 2001, which was a continuation-in part of U.S. patent application Ser. No. 09/909,492, filed Jul. 19, 2001, which was a continuation-in-part of U.S. patent application Ser. No. 09/608,706, filed Jun. 30, 2000 (now issued U.S. Pat. No. 6,303,316), which claimed the benefit under 35 U.S.C. §119(e) of provisional Patent Application Serial Nos. 60/142,301, filed Jul. 2, 1999 and 60/199,620, filed Apr. 25, 2000.

### STATEMENT REGARDING FEDERALLY SPONSORED RESEARCH OR DEVELOPMENT

The Federal Government has rights to use the present invention pursuant to contract F41624-00-D-7000 awarded by the Department of the Air Force.

### BACKGROUND OF THE INVENTION

1. Field of the Invention

The present invention relates to the field of detection of biological agents using novel compositions, methods and apparatus comprising one or more nucleic acid ligands operably coupled to an organic semiconductor. More particularly, the present invention relates to the production and use of nucleic acid ligands against Shiga toxin and/or Shiga-like toxin.

2. Description of Related Art

There is a great need for the development of methods, compositions and apparatus capable of detecting and identifying known or unknown chemical and biological agents (herein referred to as analytes), which include but are not limited to nucleic acids, proteins, illicit drugs, explosives, toxins, pharmaceuticals, carcinogens, poisons, allergens, contaminants, pathogens and infectious agents.

As one skilled in the art will readily appreciate, any method, technique or device capable of such detection and identification would have numerous medical, industrial forensic and military applications. For instance, such methods, techniques and devices could be employed in the diagnosis and treatment of disease, to develop new compounds for pharmaceutical, medical or industrial purposes, or to identify chemical and biological warfare agents.

Current methods, techniques and devices that have been applied to identification of chemical and biological analytes typically involve capturing the analyte through the use of a non-specific solid surface or through capture deoxyribonucleic acids (DNA) or antibodies. A number of known binding agents must then be applied, particularly in the case of biological analytes, until a binding agent with a high degree of affinity for the analyte is identified. A labeled antiligand (e.g., labeled DNA or labeled antibodies) must be applied, where the antiligand causes, for example, the color or fluorescence of the analyte to change if the binding agent exhibits affinity for the analyte (i.e., the binding agent binds with the analyte). The analyte may be identified by studying which of the various binding agents exhibited the greatest degree of affinity for the analyte.

There are a number of problems associated with current methods of chemical and biological agent identification. It takes a great deal of time and effort to repetitiously apply each of the known labeled antiligands, until an antiligand exhibiting a high degree of affinity is found. Accordingly, these techniques are not conducive to easy automation. Current methods are also not sufficiently robust to work in the heat, dust, humidity or other environmental conditions that might be encountered, for example, on a battlefield or in a food processing plant. Portability and ease of use are also problems seen with current methods for chemical and biological agent identification.

Within the field of biological warfare, there is a great need for a rapid, sensitive method to detect and identify Shiga toxin and/or Shiga-like toxin. Shiga toxin and/or Shiga-like toxin are highly pathogenic biological agents that are relatively simple to produce and distribute in the field. Present methods for detection of Shiga toxin and/or Shiga-like toxin are not sufficiently rapid, sensitive, and robust to allow early detection of exposure to Shiga toxin and/or Shiga-like toxin under field conditions, such as might be encountered on a battlefield. No good method presently exists for neutralization of Shiga toxin and/or Shiga-like toxin under field conditions.

### SUMMARY OF THE INVENTION

The present invention fulfills an unresolved need in the art, by providing methods, compositions and apparatus for the production of nucleic acid ligands capable of binding to, identifying and/or neutralizing Shiga toxin and/or Shiga-like toxin. The methods and compositions disclosed herein provide substantial improvements over earlier methods for detection of Shiga toxin and/or Shiga-like toxin (e.g., Donohue-Rolfe et al., 1986, J. Clin. Microbiol. 24:65-68; U.S. Pat. No. 5,955,293).

Some embodiments of the invention concern methods of preparing nucleic acid ligands against Shiga toxin and/or Shiga-like toxin, comprising obtaining a pool of nucleic acid ligands, contacting the ligands with Shiga toxin and/or Shiga-like toxin, separating and obtaining ligands that bind to the toxin. In certain embodiments, an iterative procedure is used that repeats the steps of contacting nucleic acid ligands with Shiga toxin and/or Shiga-like toxin and separating ligands that bind to the toxin. The nucleic acid ligand sequences that bind to toxin may be amplified before each round of selection. Through repeated iterations, ligands with high affinity and/or specificity for Shiga toxin and/or Shiga-like toxin may be obtained. Other embodiments concern nucleic acid ligands against Shiga toxin and/or Shiga-like toxin made by the disclosed methods.

In certain embodiments, the nucleic acid ligands may be attached to various objects, such as organic semiconductors and/or magnetic beads. Non-limiting examples of organic semiconductors of use in the disclosed methods include diazotyrosine (DAT) and diazoluminomelanin (DALM). In various embodiments, the organic semiconductor may be attached to the nucleic acid ligand by either covalent or non-covalent interaction.

In other embodiments, nucleic acid ligands that bind to Shiga toxin and/or Shiga-like toxin may be separated using nitrocellulose filter binding. In particular embodiments, nucleic acid ligands that bind to nitrocellulose filters in the absence of Shiga toxin and/or Shiga-like toxin may first be separated from the pool of nucleic acid ligands by exposure to a nitrocellulose filter.

In still other embodiments, the pool of nucleic acid ligands may comprise random 40-mers, attached at their 5′ and 3′ end to selected primer binding sequences. Such primer binding sequences facilitate the amplification of the nucleic acid ligand sequences by polymerase chain reaction (PCR™) or other amplification techniques. In certain embodiments, the primers and/or nucleic acid ligands may be attached to biotin moieties, for example to facilitate separation of single-stranded DNA (ssDNA) for use as nucleic acid ligands.

Particular embodiments of the invention concern nucleic acid ligands comprising at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 contiguous nucleotides having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 and SEQ ID NO:11. Such ligands may also comprise additional nucleotide sequences, such as primer sequences, restriction endonuclease recognition sequences, promoter sequences and other such sequences known in the art. The only requirement is that any such additional nucleotide sequences do not interfere with binding of the nucleic acid ligand to Shiga toxin and/or Shiga-like toxin. In certain embodiments, the disclosed nucleic acid ligands may be incorporated into vectors and/or attached to organic semiconductors.

Other embodiments of the invention concern methods of detecting and/or neutralizing Shiga toxin and/or Shiga-like toxin, using nucleic acid ligands that bind to Shiga toxin and/or Shiga-like toxin. In particular embodiments, neutralization may occur by attaching an organic semiconductor to one or more nucleic acid ligands that bind to Shiga toxin and/or Shiga-like toxin, exposing Shiga toxin and/or Shiga-like toxin to the nucleic acid ligand and organic semiconductor, and activating the organic semiconductor. Activation may involve exposure to a variety of activating agents, such as sunlight, heat, laser radiation, ultraviolet radiation, infrared radiation, radiofrequency radiation, microwave radiation or pulse corona discharge. Activation of the organic semiconductor/nucleic acid ligand couplet results in absorption of energy that may be transmitted to the toxin, inactivating or destroying it. (See U.S. Pat. No. 6,303,316 and U.S. patent application Ser. No. 10/291,336, filed Nov. 8, 2002, each incorporated herein by reference in its entirety.)

Detection of Shiga toxin and/or Shiga-like toxin will generally involve preparing at least one nucleic acid ligand that binds to Shiga toxin and/or Shiga-like toxin, exposing a sample to the nucleic acid ligand and detecting Shiga toxin and/or Shiga-like toxin bound to the nucleic acid ligand. In certain embodiments the nucleic acid ligand may be labeled, for example with an organic semiconductor. However, the invention is not limited by the method of detection and any method of detecting analytes known in the art may be used with nucleic acid ligands that bind Shiga toxin and/or Shiga-like toxin.

In still other embodiments, nucleic acid ligands that bind to Shiga toxin and/or Shiga-like toxin may be attached to an object, for example magnetic beads, and distributed in an environment suspected of containing Shiga toxin and/or Shiga-like toxin. The attached ligands may be collected, for example using a magnet and analyzed for the presence of bound Shiga toxin and/or Shiga-like toxin.

In preferred embodiments, the nucleic acid ligand is DNA, although it is contemplated within the scope of the invention that other nucleic acids comprised of RNA or synthetic nucleotide analogs could be utilized as well. In certain embodiments, the nucleic acid ligand sequences are random, or may be generated from libraries of random DNA sequences. In other embodiments, the nucleic acid ligand sequences may not be random, but may rather be designed to react with specific target analytes. In some embodiments, the nucleic acid ligand sequences may be aptamers (Lorsch and Szostak, In: Combinatorial Libraries: Synthesis, Screening and Application Potential. (R. Cortese, ed.) Walter de Gruyter Publishing Co., New York, pp. 69-86, 1996; Jayasena, Clin. Chem. 45: 1628-1650, 1999; U.S. Pat. Nos. 5,270,163; 5,567,588; 5,650,275; 5,670,637; 5,683,867; 5,696,249; 5,789,157; 5,843,653; 5,864,026; 5,989,823 and 6,242,246, each incorporated herein by reference).

In certain embodiments, the analyte to be detected may be added in the form of a complex mixture that may include, for example, aqueous or organic solvent, proteins, lipids, nucleic acids, detergents, particulates, intact cells, bacteria, viruses and other components. In other embodiments, the analyte may be partially or fully purified before detection. In particularly preferred embodiments, the analyte is Shiga toxin and/or Shiga-like toxin.

In certain embodiments, a recognition complex system, comprising two or more recognition complexes, each recognition complex comprising a nucleic acid ligand attached to an organic semiconductor, may be used in methods of detecting an analyte. After the analyte is contacted with the recognition complexes, certain recognition complexes will bind the analyte, while others will not. Binding of analyte to a recognition complex may be detected by changes in the photochemical properties of the nucleic acid ligand/organic semiconductor couplet upon binding to the analyte. Non-limiting examples of photochemical signals include fluorescent, phosphorescent or luminescent signals or changes in color. The degree to which the photochemical properties change is a function of the degree to which the nucleic acid ligand binds the analyte. Accordingly, the photochemical changes that occur across all of the recognition complexes, when taken as a whole, can be used as a unique signature to identify the analyte.

To facilitate detection of such photochemical changes, the recognition complex system may be associated with a detection unit operably coupled to the recognition complexes. Non-limiting examples of detection units include a charge coupled device (CCD), a CCD camera, a photomultiplier tube, a spectrophotometer or a fluorometer. The recognition complex system may also be associated with system memory for storing photochemical signals, as well as a data processing unit.

In certain embodiments, the recognition complexes may be attached to a surface, such as a Langmuir-Blodgett film, functionalized glass, plastic, germanium, silicon, PTFE, polystyrene, gallium arsenide, gold, silver, nitrocellulose or other membrane, nylon, glass bead, magnetic bead or PVP. In some embodiments, the recognition complexes may be distributed across the surface of a chip so as to form an array. In other embodiments, the recognition complexes may be attached to a surface for use in a flow cell apparatus. In particular embodiments, recognition complexes may be incorporated into a card or badge.

The skilled artisan will realize that magnetic beads may be used for separating recognition complexes that bind to the analyte from recognition complexes that do not bind the analyte. In one embodiment, a magnetic flow cell, such as is described in U.S. Pat. No. 5,972,721 (incorporated herein by reference), could be used in conjunction with the recognition complex system to identify and separate analyte-binding recognition complexes from recognition complexes that do not bind the analyte.

In certain embodiments, flow cytometry may be used to separate recognition complexes that bind to an analyte from those that do not bind. In such embodiments, the recognition complex may be attached to a glass or other bead. Nucleic acid ligands that bind to the Shiga toxin and/or Shiga-like toxin may be sorted, for example, by screening particles for organic semiconductor-associated fluorescence in a flow cytometer.

The skilled artisan will realize that the methods disclosed herein are not limited to methods of preparation and use of nucleic acid ligands against Shiga toxin and/or Shiga-like toxin, but rather are applicable to a variety of analytes, including all other toxins and/or venoms.

### BRIEF DESCRIPTION OF THE DRAWINGS

No drawings are necessary for the understanding of the subject matter of the present invention.

### DESCRIPTION OF ILLUSTRATIVE EMBODIMENTS

Definitions

As used herein, “a” or “an” may mean one or more than one of an item.

“Nucleic acid” means either DNA, RNA, single-stranded, double-stranded or triple stranded and any chemical modifications thereof. Virtually any modification of the nucleic acid is contemplated by this invention. Non-limiting examples of nucleic acid modifications are discussed in further detail below. “Nucleic acid” encompasses, but is not limited to, oligonucleotides and polynucleotides. “Oligonucleotide” refers to at least one molecule of between about 3 and about 100 nucleotides in length. “Polynucleotide” refers to at least one molecule of greater than about 100 nucleotides in length. These terms generally refer to at least one single-stranded molecule, but in certain embodiments also encompass at least one additional strand that is partially, substantially or fully complementary in sequence. Thus, a nucleic acid may encompass at least one double-stranded molecule or at least one triple-stranded molecule that comprises one or more complementary strand(s). A “nucleic acid” may be of almost any length, from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, 100, 110, 120, 130, 140, 150, 160, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10000, 15000, 20000 or even more bases in length.

“Nucleic acid ligand” means a non-naturally occurring nucleic acid having an effect on a target. An effect includes, but is not limited to, binding to the target, catalytically changing the target, reacting with the target in a way that modifies or alters the target or the functional activity of the target, covalently attaching to the target, facilitating the reaction between the target and another molecule, and neutralizing the target. In a preferred embodiment, the action is specific binding to a target molecule, such as Shiga toxin and/or Shiga-like toxin. “Nucleic acid ligand” specifically excludes nucleic acids that bind to another nucleic acid through a mechanism that predominantly depends on Watson/Crick base pairing.

“Analyte,” “target” and “target analyte” mean any compound or aggregate of interest. Non-limiting examples of analytes include a protein, polypeptide, peptide, carbohydrate, polysaccharide, glycoprotein, lipid, hormone, receptor, antigen, allergen, antibody, substrate, metabolite, cofactor, inhibitor, drug, pharmaceutical, nutrient, toxin, cholera toxin, Shiga toxin, Shiga-like toxin, poison, explosive, pesticide, chemical warfare agent, biohazardous agent, anthrax spore, prion, radioisotope, vitamin, heterocyclic aromatic compound, carcinogen, mutagen, narcotic, amphetamine, barbiturate, hallucinogen, waste product, contaminant or other molecule. Molecules of any size can serve as targets. “Analytes” are not limited to single molecules, but may also comprise complex aggregates of molecules, such as a virus, bacterium, spore, mold, yeast, algae, amoebae, dinoflagellate, unicellular organism, pathogen, cell or infectious agent. In certain embodiments, cells exhibiting a particular characteristic or disease state, such as a cancer cell, may be target analytes. Virtually any chemical or biological effector would be a suitable target. In particularly preferred embodiments, the analyte is Shiga toxin and/or Shiga-like toxin.

Non-limiting examples of infectious agents within the meaning of “analyte” are listed in Table 1 below.

TABLE 1
Non-limiting Exemplary Infectious Agents

Actinobacillus spp.
Actinomyces spp.
Adenovirus (types 1, 2, 3, 4, 5 et 7)
Adenovirus (types 40 and 41)
Aerococcus spp.
Aeromonas hydrophila
Ancylostoma duodenale
Angiostrongylus cantonensis
Ascaris lumbricoides
Ascaris spp.
Aspergillus spp.
Bacillus anthracis
Bacillus cereus
Bacteroides spp.
Balantidium coli
Bartonella bacilliformis
Blastomyces dermatitidis
Bluetongue virus
Bordetella bronchiseptica
Bordetella pertussis
Borrelia burgdorferi
Branhamella catarrhalis
Brucella spp.
B. abortus
B. canis,
B. melitensis
B. suis
Brugia spp.
Burkholderia mallei
Burkholderia pseudomallei
Campylobacter fetus subsp. fetus
Campylobacter jejuni
C. coli
C. fetus subsp. jejuni
Candida albicans
Capnocytophaga spp.
Chlamydia psittaci
Chlamydia trachomatis
Citrobacter spp.
Clonorchis sinensis
Clostridium botulinum
Clostridium difficile
Clostridium perfringens
Clostridium tetani
Clostridium spp.
Coccidioides immitis
Colorado tick fever virus
Corynebacterium diphtheriae
Coxiella burnetii
Coxsackievirus
Creutzfeldt-Jakob agent, Kuru agent
Crimean-Congo hemorrhagic fever virus
Cryptococcus neoformans
Cryptosporidium parvum
Cytomegalovirus
Dengue virus (1, 2, 3, 4)
Diphtheroids
Eastern (Western) equine
encephalitis virus
Ebola virus
Echinococcus granubosus
Echinococcus multibocularis
Echovirus
Edwardsiella tarda
Entamoeba histolytica
Enterobacter spp.
Enterovirus 70
Epidermophyton floccosum,
Microsporum spp. Trichophyton spp.
Epstein-Barr virus
Escherichia coli, enterohemorrhagic
Escherichia coli, enteroinvasive
Escherichia coli, enteropathogenic
Escherichia coli, enterotoxigenic
Fasciola hepatica
Francisella tularensis
Fusobacterium spp.
Gemella haemolysans
Giardia lamblia
Giardia spp.
Haemophilus ducreyi
Haemophilus influenzae (group b)
Hantavirus
Hepatitis A virus
Hepatitis B virus
Hepatitis C virus
Hepatitis D virus
Hepatitis E virus
Herpes simplex virus
Herpesvirus simiae
Histoplasma capsulatum
Human coronavirus
Human immunodeficiency virus
Human papillomavirus
Human rotavirus
Human T-lymphotrophic virus
Influenza virus
Junin virus/Machupo virus
Klebsiella spp.
Kyasanur Forest disease virus
Lactobacillus spp.
Legionella pneumophila
Leishmania spp.
Leptospira interrogans
Listeria monocytogenes
Lymphocytic choriomeningitis virus
Marburg virus
Measles virus
Micrococcus spp.
Moraxella spp.
Mycobacterium spp.
Mycobacterium tuberculosis, M. bovis
Mycoplasma hominis, M. orale, M.
salivarium, M. fermentans
Mycoplasma pneumoniae
Naegleria fowleri
Necator americanus
Neisseria gonorrhoeae
Neisseria meningitidis
Neisseria spp.
Nocardia spp.
Norwalk virus
Omsk hemorrhagic fever virus
Onchocerca volvulus
Opisthorchis spp.
Parvovirus B19
Pasteurella spp.
Peptococcus spp.
Peptostreptococcus spp.
Plesiomonas shigelloides
Powassan encephalitis virus
Proteus spp.
Pseudomonas spp.
Rabies virus
Respiratory syncytial virus
Rhinovirus
Rickettsia akari
Rickettsia prowazekii, R. canada
Rickettsia rickettsii
Ross river virus/O'Nyong-Nyong virus
Rubella virus
Salmonella choleraesuis
Salmonella paratyphi
Salmonella typhi
Salmonella spp.
Schistosoma spp.
Scrapie agent
Serratia spp.
Shigella spp.
Sindbis virus
Sporothrix schenckii
St. Louis encephalitis virus
Murray Valley encephalitis virus
Staphylococcus aureus
Streptobacillus moniliformis
Streptococcus agalactiae
Streptococcus faecalis
Streptococcus pneumoniae
Streptococcus pyogenes
Streptococcus salivarius
Taenia saginata
Taenia solium
Toxocara canis, T. cati
Toxoplasma gondii
Treponema pallidum
Trichinella spp.
Trichomonas vaginalis
Trichuris trichiura
Trypanosoma brucei
Ureaplasma urealyticum
Vaccinia virus
Varicella-zoster virus
Venezuelan equine encephalitis
Vesicular stomatitis virus
Vibrio cholerae, serovar 01
Vibrio parahaemolyticus
Wuchereria bancrofti
Yellow fever virus
Yersinia enterocolitica
Yersinia pseudotuberculosis
Yersinia pestis

“Binding” refers to an interaction between a target and a nucleic acid ligand, resulting in a sufficiently stable complex so as to permit separation of nucleic acid ligand:target complexes from uncomplexed nucleic acid ligands under given binding or reaction conditions. Binding is mediated through hydrogen bonding, electrostatic interaction, hydrophobic interaction, Van der Walls forces or other molecular forces. In certain embodiments, binding may be covalent, for example where the nucleic acid ligand or analyte contains a photoreactive or chemically reactive moiety to promote covalent attachment of ligand and analyte. In alternative embodiments, binding may be non-covalent.

“Organic semiconductor” means a conjugated (alternating double and single bonded) organic compound in which regions of electrons and the absence of electrons (holes or positive charges) can move with varying degrees of difficulty through the aligned conjugated system (varying from insulator to conductor). An organic semiconductor may be thought of as the organic equivalent of a metal. Organic semiconductors are distinguished from metals in their spectroscopic properties. Organic semiconductors of use in the practice of the instant invention may be fluorescent, luminescent, chemiluminescent, sonochemiluminescent, thermochemiluminescent or electrochemiluminescent or may be otherwise characterized by their absorption, reflection or emission of electromagnetic radiation, including infrared, ultraviolet or visible light. In preferred embodiments, the organic semiconductor is DAT or DALM.

“Recognition complex” refers to a nucleic acid ligand that is operably coupled to an organic semiconductor. “Operably coupled” means that the nucleic acid ligand and the organic semiconductor are in close physical proximity to each other, such that binding of an analyte to the nucleic acid ligand results in a change in the properties of the organic semiconductor that is detectable as a signal. In preferred embodiments, the signal is a photochemical signal. In one preferred embodiment, the signal is a change in the fluorescence emission profile of the organic semiconductor/nucleic acid ligand couplet. Operable coupling may be accomplished by a variety of interactions, including but not limited to non-covalent or covalent binding of the organic semiconductor to the nucleic acid ligand. In another embodiment, the nucleic acid ligand may be at least partially embedded in the organic semiconductor. Virtually any type of interaction between the organic semiconductor and the nucleic acid ligand is contemplated within the scope of the present invention, so long as the binding of an analyte to the nucleic acid ligand results in a change in the photochemical properties of the organic semiconductor.

A “recognition complex system” comprises an array of recognition complexes. In preferred embodiments, the array of recognition complexes is operably coupled to a detection unit, such that changes in the photochemical properties of the organic semiconductor that result from binding of analyte to nucleic acid ligand may be detected by the detection unit. It is contemplated within the scope of the present invention that detection may be an active process or a passive process. For example, in embodiments where the array of recognition complexes is incorporated into a card or badge, the binding of analyte may be detected by a change in color of the card or badge. In other embodiments, detection occurs by an active process, such as scanning the fluorescence emission profile of an array of recognition complexes.

Shiga Toxin and Shiga-Like Toxins

Shiga toxin is a multimeric protein toxin that is produced by the bacterium Shigella dysenteriae type I (U.S. Pat. No. 5,955,293). Exposure to Shiga toxin can cause enterotoxicity, neurotoxicity, cytotoxicity, paralysis and death (Id.). These effects of the toxin are thought to be related to the pathogenic effects of Shigella infection (Id.). Among other things, Shiga toxin inhibits protein synthesis through inactivation of ribosomes (Id.). The toxin comprises one copy of an A chain peptide and five copies of a B chain peptide (Id.). The B chain binds to cell surface receptors while the A chain is responsible for at least some of the toxic effects of Shiga toxin (Id.). Methods of purification of Shiga toxin and related proteins have been reported (Id.).

Many related cytotoxins are reported to be produced by other bacterial species, such as E. coli, Vibrio, Salmonella and Campylobacter (Id.). Verotoxin is reported to be produced by E. coli strains that are associated with hemolytic uremic syndrome and hemorrhagic colitis (Id.). A cytotoxin produced by E. coli 0157:H7, a bacterial strain associated with hemorrhagic intestinal disease caused by food poisoning, was reportedly neutralized by antibodies against Shiga toxin and has been designated as a Shiga-like toxin (Id.). Different forms of toxins produced by E. coli 0157:H7 have been designated Shiga-like toxin I and II (Id.). Shiga toxin and Shiga-like toxin I are almost identical in amino acid sequence, while Shiga-like toxin I and II only share 56% amino acid sequence homology (Id.). Other toxins related to Shiga-like toxin II are also known (Id.). All of the Shiga toxin and Shiga-like toxin proteins may be used in the claimed methods.

Nucleic Acid Ligands

Nucleic acid ligands within the scope of the present invention may be made by any technique known to one in the art. Non-limiting examples of nucleic acid ligands include synthetic oligonucleotides. Oligonucleotides may be synthesized using phosphotriester, phosphite or phosphoramidite chemistry and solid phase techniques (EP 266,032, incorporated herein by reference) or via deoxynucleoside H-phosphonate intermediates (Froehler et al., Nucleic Acids Research, 14:5399-5467, 1986, and U.S. Pat. No. 5,705,629, each incorporated herein by reference). Examples of enzymatically produced nucleic acid ligands include those produced by amplification reactions such as PCR™ (e.g., U.S. Pat. No. 4,683,202 and U.S. Pat. No. 4,683,195, each incorporated herein by reference), or as disclosed in U.S. Pat. No. 5,645,897, incorporated herein by reference. Examples of a biologically produced nucleic acid ligand include recombinant nucleic acid production in living cells, such as recombinant DNA vector production in bacteria (e.g., Sambrook et al. In: Molecular Cloning: A Laboratory Manual, 2d Ed., Cold Spring Harbor Laboratory Press, Cold Spring Harbor, N.Y., 1989).

In general, a minimum of approximately 3 nucleotides, preferably at least 5 nucleotides, are necessary to effect specific binding of nucleic acid ligands to a target. However, the size of the nucleic acid ligands is not limiting and binding sequences of 10, 15, 20, 25, 20, 25, 40, 45, 50, 60, 70, 80, 90 or 100 nucleotides or longer may be used. In preferred embodiments, the binding sequences are 40 nucleotides long. The specifically binding nucleotides may be attached to flanking regions and otherwise derivatized. In preferred embodiments of the invention, the analyte-binding sequences will be flanked by known, amplifiable sequences, facilitating the amplification of the nucleic acid ligands by PCR or other amplification techniques. In a further embodiment, the flanking sequence may comprise a specific sequence that preferentially recognizes or binds a moiety to enhance the immobilization of the ligand to a substrate.

The nucleic acid ligands found to bind to the targets may be isolated, sequenced, and/or amplified or synthesized as conventional DNA or RNA molecules. Alternatively, nucleic acid ligands of interest may comprise modified oligomers. Any of the hydroxyl groups ordinarily present in nucleic acid ligands may be replaced by phosphonate groups, phosphate groups, protected by a standard protecting group, or activated to prepare additional linkages to other nucleotides, or may be conjugated to solid supports. The 5′ terminal OH is conventionally free but may be phosphorylated. Hydroxyl group substituents at the 3′ terminus may also be phosphorylated. The hydroxyls may be derivatized by standard protecting groups. One or more phosphodiester linkages may be replaced by alternative linking groups. These alternative linking groups include, exemplary embodiments wherein P(O)O is replaced by P(O)S, P(O)NR₂, P(O)R, P(O)OR′, CO, or CNR₂, wherein R is H or alkyl (1-20C) and R′ is alkyl (1-20C); in addition, this group may be attached to adjacent nucleotides through O or S. Not all linkages in an oligomer need to be identical.

The nucleic acid ligands used as starting materials in the process of the invention to determine specific binding sequences may be single-stranded or double-stranded DNA or RNA. In a preferred embodiment, the sequences are single-stranded DNA. The use of DNA eliminates the need for conversion of RNA to DNA by reverse transcriptase prior to PCR amplification. Furthermore, DNA is less susceptible to nuclease degradation than RNA. In preferred embodiments, the starting nucleic acid ligand will contain a randomized sequence portion, generally including from about 10 to 400 nucleotides, more preferably 20 to 100 nucleotides. The randomized sequence is flanked by primer sequences that permit the amplification of nucleic acid ligands found to bind to the analyte. The flanking sequences may also contain other convenient features, such as restriction sites. These primer hybridization regions generally contain 10 to 30, more preferably 15 to 25, bases of known sequence.

Both the randomized portion and the primer hybridization regions of the initial oligomer population are preferably constructed using conventional solid phase techniques. Such techniques are well known in the art. Nucleic acid ligands may also be synthesized using solution phase methods such as triester synthesis, known in the art. For synthesis of the randomized regions, mixtures of nucleotides at the positions where randomization is to occur are added during synthesis. Any degree of randomization may be employed. Some positions may be randomized by mixtures of only two or three bases rather than the conventional four. Randomized positions may alternate with those that have been specified.

Nucleic acid ligands within the scope of the present invention may comprise one or more nucleotide mimics or derivatives. Nucleotide mimics and derivatives are well known in the art, and have been described in exemplary references such as, for example, Scheit, Nucleotide Analogs (John Wiley, New York, 1980). These include, but are not limited to, purines and pyrimidines substituted with one or more alkyl, carboxyalkyl, amino, hydroxyl, halogen (i.e. fluoro, chloro, bromo, or iodo), thiol, or alkylthiol groups. The alkyl substituents may comprise from about 1, 2, 3, 4, or 5, to about 6 carbon atoms.

Examples of purines and pyrimidines include deazapurines, 2,6-diaminopurine, 5fluorouracil, xanthine, hypoxanthine, 8-bromoguanine, 8-chloroguanine, bromothymine, 8-aminoguanine, 8-hydroxyguanine, 8-methylguanine, 8-thioguanine, azaguanines, 2-aminopurine, 5-ethylcytosine, 5-methylcytosine, 5-bromouracil, 5-ethyluracil, 5-iodouracil, 5-chlorouracil, 5-propyluracil, thiouracil, 2-methyladenine, methylthioadenine, N,N-dimethyladenine, azaadenines, 8-bromoadenine, 8-hydroxyadenine, 6-hydroxyaminopurine, 6-thiopurine, 4-(6-aminohexyl/cytosine), and the like. A list of exemplary purine and pyrimidine derivatives and mimics is provided in Table 2.

TABLE 2
Purine and Pyrimidine Derivatives or Mimics

| Abbr. | Modified base description | Abbr. | Modified base description |
|---|---|---|---|
| ac4c | 4-acetylcytidine | mam5s2u | 5-methoxyaminomethyl-2- |
| | | | thiouridine |
| chm5u | 5- | man q | Beta,D-mannosylqueosine |
| | (carboxyhydroxylmethyl)uridine | | |
| Cm | 2′-O-methylcytidine | mcm5s2u | 5-methoxycarbonylmethyl-2- |
| | | | thiouridine |
| cmnm5s2u | 5-carboxymethylaminomethyl-2- | mcm5u | 5-methoxycarbonylmethyluridine |
| | thioridine | | |
| cmnm5u | 5- | mo5u | 5-methoxyuridine |
| | carboxymethylaminomethyluridine | | |
| D | Dihydrouridine | ms2i6a | 2-methylthio-N6- |
| | | | isopentenyladenosine |
| Fm | 2′-O-methylpseudouridine | ms2t6a | N-((9-beta-D-ribofuranosyl-2- |
| | | | methylthiopurine-6- |
| | | | yl)carbamoyl)threonine |
| gal q | beta,D-galactosylqueosine | mt6a | N-((9-beta-D-ribofuranosylpurifle- |
| | | | 6-yl)N-methyl-carbamoyl)threonine |
| Gm | 2′-O-methylguanosine | mv | Uridine-5-oxyacetic acid |
| | | | methylester |
| I | Inosine | o5u | Uridine-5-oxyacetic acid (v) |
| i6a | N6-isopentenyladenosine | osyw | Wybutoxosine |
| m1a | 1-methyladenosine | p | Pseudouridine |
| m1f | 1-methylpseudouridine | q | Queosine |
| m1g | 1-methylguanosine | s2c | 2-thiocytidine |
| m1I | 1-methylinosine | s2t | 5-methyl-2-thiouridine |
| m22g | 2,2-dimethylguanosine | s2u | 2-thiouridine |
| m2a | 2-methyladenosine | s4u | 4-thiouridine |
| m2g | 2-methylguanosine | t | 5-methyluridine |
| m3c | 3-methylcytidine | t6a | N-((9-beta-D-ribofuranosylpurine- |
| | | | 6-yl)carbamoyl)threonine |
| m5c | 5-methylcytidine | tm | 2′-O-methyl-5-methyluridine |
| m6a | N6-methyladenosine | um | 2′-O-methyluridine |
| m7g | 7-methylguanosine | yw | Wybutosine |
| mam5u | 5-methylaminomethyluridine | x | 3-(3-amino-3- |
| | | | carboxypropyl)uridine, (acp3)u |

An example of a nucleic acid ligand comprising nucleoside or nucleotide derivatives and mimics is a “polyether nucleic acid”, described in U.S. Pat. No. 5,908,845, incorporated herein by reference, wherein one or more nucleobases are linked to chiral carbon atoms in a polyether backbone. Another example of a nucleic acid ligand is a “peptide nucleic acid”, also known as a “PNA” (i.e., U.S. Pat. No. 5,539,082), “peptide-based nucleic acid mimics” or “PENAMs”, disclosed in U.S. Pat. Nos. 5,786,461, 5,891,625, 5,773,571, 5,766,855, 5,736,336, 5,719,262, 5,714,331, 5,539,082, and WO 92/20702, each of which is incorporated herein by reference.

Peptide nucleic acids generally have enhanced sequence specificity, binding properties, and resistance to enzymatic degradation in comparison to molecules such as DNA and RNA (Egholm et al., Nature, 365:566, 1993). In addition, U.S. Pat. Nos. 5,766,855, 5,719,262, 5,714,331 and 5,736,336 describe PNAs comprising nucleobases and alkylamine side chains with further improvements in sequence specificity, solubility and binding affinity. These properties promote double or triple helix formation between a target and the PNA. The skilled artisan will realize that the claimed nucleic acid ligands are not limited to the examples disclosed herein, but may include nucleobases, nucleotides and nucleic acids produced by any other means known in the art.

Production of Nucleic Acid Ligands by SELEX

An exemplary method for preparing nucleic acid ligands against various analytes is known as SELEX (e.g., U.S. Pat. Nos. 5,475,096 and 5,270,163, each incorporated by reference). The SELEX method involves selection from a mixture of candidate nucleic acid ligands and step-wise iterations of binding, partitioning and amplification, using the same general selection scheme, to achieve a selected degree of binding affinity and selectivity. Starting from a mixture of candidate nucleic acid ligands, preferably comprising a segment of randomized sequence, the method includes the following. Contacting the mixture with the target under conditions favorable for binding. Partitioning unbound nucleic acid ligands from those nucleic acid ligands that have bound specifically to target analyte. Dissociating the nucleic acid ligand-analyte complexes. Amplifying the nucleic acid ligands dissociated from the nucleic acid ligand-analyte complexes to yield mixture of nucleic acid ligands that preferentially bind to the analyte. Reiterating the steps of binding, partitioning, dissociating and amplifying through as many cycles as appropriate to yield highly specific, nucleic acid ligands that bind with high affinity to the target analyte.

In the SELEX process, a candidate mixture of nucleic acid ligands of differing sequence is prepared. The candidate mixture generally includes regions of fixed sequences (i.e., each of the nucleic acid ligands contains the same sequences) and regions of randomized sequences. The fixed sequence regions may be selected to: (a) assist in the amplification steps; (b) mimic a sequence known to bind to the target; or (c) promote the formation of a given structural arrangement of the nucleic acid ligands. The randomized sequences may be totally randomized (i.e., the probability of finding a given base at any position being one in four) or only partially randomized (i.e., the probability of finding a given base at any location can be any level between 0 and 100 percent).

Because only a small number of sequences corresponding to the highest affinity nucleic acid ligands may be present in the starting pool, it may be necessary to set the partitioning criteria so that a significant amount of nucleic acid ligands in the mixture (approximately 5-50%) are retained during partitioning. Those nucleic acid ligands selected during partitioning as having higher affinity for the target may be amplified to create a new candidate mixture that is enriched in higher affinity nucleic acid ligands.

By repeating the partitioning and amplifying steps, each round of candidate mixture contains fewer and fewer weakly binding sequences. The average degree of specificity and affinity of the nucleic acid ligands to target will generally increase with each cycle. The SELEX process can ultimately yield a mixture containing one or a small number of nucleic acid ligands having the highest specificity and affinity for the target analyte.

Nucleic acid ligands produced for SELEX may be generated on a commercially available DNA synthesizer (e.g., Applied Biosystems, Foster City, Calif.). The random region is produced by mixing equimolar amounts of each nitrogenous base (A, C, G, and T) at each position to create a large number of permutations (i.e., 4ⁿ, where “n” is the oligonucleotide chain length) in a very short segment. Thus a randomized 40 mer library may consist of 4³⁰ or maximally 10²⁴ different nucleic acid ligands. Because of constraints on the amount of nucleic acids that may be synthesized and screened, the actual number of different nucleic acid sequences present in the starting pool may be substantially lower than the theoretical maximum. The random region may be flanked by two short primer regions to enable amplification of the subset of nucleic acid ligands that bind to the target analyte.

Production of Nucleic Acid Ligands Using Magnetic Beads

In alternative embodiments of the invention, nucleic acid ligands may be attached to magnetic beads. In a preferred embodiment, each nucleic acid ligand molecule attached to the same magnetic bead will have the same sequence. In other embodiments, the nucleic acid ligand molecules attached to a single bead may have different sequences. In certain preferred embodiments, the nucleic acid ligands will be attached to an organic semiconductor.

The skilled artisan will realize that use of magnetic bead technology would facilitate certain applications of the invention, such as the iterative process for producing nucleic acid ligands of higher specificity and greater binding affinity for the analyte. With magnetic bead technology, the individual recognition complexes are more easily manipulated and separated according to their characteristics. For example, recognition complexes that bind to the analyte may be separated from recognition complexes that do not bind to the analyte by using a magnetic flow cell or filter block, as disclosed in U.S. Pat. No. 5,972,721, incorporated herein by reference.

Nucleic acid ligands of random or non-random sequence may be synthesized or amplified and attached to magnetic beads, preferably with organic semiconductor. The array of beads may be added to a magnetic bead mixer and analyte added and allowed to bind to the nucleic acid ligands. The mixture may then be transferred to a photochemical cell with a magnetic electrode, where the mixture may be exposed to ultraviolet or other irradiation. A CCD, photomultiplier tube, digital camera or other detection device may be used to obtain absorption or emission spectra. Binding of analyte will result in characteristic changes in the photochemical properties of individual recognition complexes. Although the suspension of recognition complexes in the bead mixer is random, the use of a magnetic electrode in the photochemical cell will provide a spatial distribution of recognition complexes. Beads will deposit and separate on the surface of the magnetic electrode according to their accumulated mass (from binding analyte). This spatial distribution, along with the detected photochemical changes, may be analyzed to produce a unique signature that can be used to identify the analyte.

After detection, the recognition complexes may be transferred to a magnetic filter, where recognition complexes that bind to the analyte may be separated from those that do not bind analyte. The recognition complexes that do not bind analyte may be transferred to a recycle bin, where the nucleic acid ligands may be detached from the magnetic beads. The magnetic beads may be disposed of or recycled for attachment to new nucleic acid ligands. Those recognition complexes that bind to the analyte may be transferred to a PCR cycler, where the nucleic acid ligand sequences may be amplified. The new nucleic acid ligand sequences may be attached to magnetic beads and transferred to the magnetic bead mixer for another iteration of the process.

Processes for the coupling of molecules to magnetic beads or a magnetite substrate are known in the art (i.e. U.S. Pat. Nos. 4,695,393, 3,970,518, 4,230,685, and 4,677,055, incorporated herein by reference). Alternatively, an organic semiconductor may be attached directly to the magnetic bead. Nucleic acid ligands may be attached to the organic semiconductor by electrostatic interaction with magnesium ion, or by covalent attachment such as by silane coupling. Various silane couplings applicable to magnetic beads are discussed in U.S. Pat. No. 3,652,761. Procedures for silanization are generally known in the art (e.g., Weetall, in: Methods in Enzymology, K. Mosbach, ed., 44:134-148, 1976 and U.S. Pat. Nos. 3,933,997 and 3,652,761).

While large magnetic particles (mean diameter in solution greater than 10 μm) can respond to weak magnetic fields and magnetic field gradients, they tend to settle rapidly. In preferred embodiments, the magnetic beads are less than 10 μm in diameter. Although particles of any size may be used within the scope of the invention, preferred magnetic particles are between about 0.1 and about 1.5 μm diameter. Particles with mean diameters in this range can be produced with a surface area as high as about 100 to 150 m²/gm, which provides a high capacity for bioaffinity adsorbent coupling. Magnetic particles of this size range overcome the rapid settling problems of larger particles, but obviate the need for large magnets to generate the magnetic fields and magnetic field gradients required to separate smaller particles. Magnets used to effect separations of the magnetic particles of this invention need only generate magnetic fields between about 100 and about 1000 Oersteds.

Ferromagnetic materials become permanently magnetized in response to magnetic fields. Superparamagnetic particles respond to magnetic field gradients, but do not become permanently magnetized. Crystals of magnetic iron oxides may be either ferromagnetic or superparamagnetic, depending on the size of the crystals. Superparamagnetic oxides of iron generally result when the crystal is less than about 300 angstroms (Å) in diameter, while larger crystals generally have a ferromagnetic character. In preferred embodiments, superparamagnetic particles are used.

Methods of preparing magnetic particles are known in the art (e.g., U.S. Pat. No. 4,267,234, incorporated herein by reference). The method may comprise precipitating metal salts in base to form fine magnetic metal oxide crystals, redispersing and washing the crystals in water and in an electrolyte. Magnetic separations may be used to collect the crystals between washes if the crystals are superparamagnetic. The crystals may then be coated with a material capable of adsorptively or covalently bonding to the metal oxide and bearing functional groups for coupling with nucleic acid ligands and/or organic semiconductor.

Production of Nucleic Acid Ligands Using Flow Cytometry

In other embodiments, the recognition complexes of interest may be non-covalently or covalently attached to non-magnetic beads, such as glass, polyacrylamide, polystyrene or latex, using the same techniques discussed above for magnetic beads. After exposure of analyte to receptor complexes, those complexes bound to analyte may be separated from unbound complexes by flow cytometry. Non-limiting examples of flow cytometry methods are disclosed in Betz et al. (Cytometry 5: 145-150, 1984), Wilson et al. (J. Immunol. Methods 107: 231-237, 1988), Scillian et al. (Blood 73: 2041-2048, 1989), Frengen et al. (Clin. Chem. 40/3: 420-425, 1994), Griffith et al. (Cytometry 25: 133-143, 1996), Stuart et al. (Cytometry 33: 414-419, 1998) and U.S. Pat. Nos. 5,853,984 and 5,948,627, incorporated herein by reference. U.S. Pat. Nos. 4,727,020, 4,704,891 and 4,599,307, incorporated herein by reference, describe the arrangement of the components comprising a flow cytometer and the general principles of its use.

In the flow cytometer, beads, cells or other particles are passed substantially one at a time through a detector, where each particle is exposed to an energy source. The energy source generally provides excitatory light of a single wavelength. The detector comprises a light collection unit, such as photomultiplier tubes or a charge coupled device, which may be attached to a data analyzer such as a computer. The beads, cells or particles can be characterized by their response to excitatory light, for example by detecting and/or quantifying the amount of fluorescent light emitted in response to the excitatory light. Changes in size due to binding of analyte to ligand can also be incorporated into sorting strategies. Beads or cells exhibiting a particular characteristic can be sorted using an attached cell sorter, such as the FACS Vantage™ cell sorter sold by Becton Dickinson Immunocytometry Systems (San Jose, Calif.).

This system is well suited to use with an organic semiconductor that has well defined fluorescent and luminescent properties. Using a flow cytometer, it is possible to separate beads, cells or particles that are associated with recognition complexes bound to analytes, from unbound complexes, by detecting the presence of and characterizing the photochemical properties of the organic semiconductor. Because those properties change upon binding of recognition complex to analyte, it is possible to separate bead-attached recognition complexes that bind to analyte from complexes that do not bind analyte.

Nucleic Acid Ligand Amplification

In certain embodiments, the nucleic acid ligands may be subjected to amplification, such as by polymerase chain reaction amplification (PCR™). Within the scope of the present invention, amplification may be accomplished by any means known in the art. Exemplary methods are disclosed below.

Primers

Primers encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred. In preferred embodiments, primers are selected that are complementary to known binding sites on the nucleic acids to be amplified. In certain alternative embodiments, random primers may be utilized. Primers may be prepared by any method known in the art, such as by standard oligonucleotide chemical synthesis.

Amplification Methods

A number of template dependent processes are known in the art. One of the best known amplification methods is polymerase chain reaction (PCR™) amplification (see Innis et al., PCR Protocols, Academic Press, Inc., San Diego Calif., 1990; U.S. Pat. Nos. 4,683,195, 4,683,202 and 4,800,159, incorporated herein by reference). In PCR, two primer sequences are prepared which are complementary to regions on opposite complementary strands of, for example, a nucleic acid ligand. An excess of deoxynucleoside triphosphates are added to a reaction mixture along with a DNA polymerase, e.g., Taq polymerase. The primers will bind to primer binding sites on the nucleic acid ligands and the polymerase will cause the primers to be extended by adding on nucleotides. By raising and lowering the temperature of the reaction mixture, the extended primers will dissociate from the nucleic acid ligand to form reaction products, excess primers will bind to the nucleic acid ligand and to the reaction products and the process is repeated.

A reverse transcriptase PCR amplification procedure may be performed in order to amplify, for example, mRNA. Methods of reverse transcribing RNA into cDNA are well known (e.g., Sambrook et al., In: Molecular Cloning: A Laboratory Manual, 2d Ed., Cold Spring Harbor Laboratory Press, Cold Spring Harbor, N.Y., 1989).

Another method for amplification is ligase chain reaction (“LCR”) (European Patent Application No. 320,308). In LCR, two complementary probe pairs are prepared, and in the presence of the nucleic acid ligand sequence, each pair will bind to opposite complementary strands of the target such that they abut. In the presence of a ligase, the two probe pairs will link to form a single unit. By temperature cycling, as in PCR, bound ligated units dissociate from the nucleic acid ligand and then serve as templates for ligation of excess probe pairs. U.S. Pat. No. 4,883,750 discloses a method similar to LCR for binding probe pairs to a nucleic acid ligand sequence.

Qbeta Replicase, disclosed in PCT Application No. PCT/US87/00880, may also be used as an amplification method. In this method, a replicative sequence of RNA that has a region complementary to that of a nucleic acid ligand is added to a sample in the presence of an RNA polymerase. The polymerase will copy the replicative sequence.

An isothermal amplification method, in which restriction endonucleases and ligases are used to achieve the amplification of nucleic acid ligand molecules that contain nucleotide 5′-[alpha-thio]-triphosphates in one strand of a restriction site may also be useful in the amplification of nucleic acid ligands in the present invention (Walker et al., Proc. Natl. Acad. Sci. USA, 89:392-396, 1992).

Strand Displacement Amplification (SDA) is another method of carrying out isothermal amplification of nucleic acid ligands, involving multiple rounds of strand displacement and synthesis, i.e., nick translation. A similar method, called Repair Chain Reaction (RCR), involves annealing several probes throughout a region targeted for amplification, followed by a repair reaction. A similar approach is used in SDA.

Still other amplification methods disclosed in GB Application No. 2 202 328, and in PCT Application No. PCT/US89/01025, may be used in accordance with the present invention. In the former application, “modified” primers are used in a PCR like, template and enzyme dependent synthesis. The primers may be modified by labeling with a capture moiety (e.g., biotin). Other nucleic acid ligand amplification procedures include transcription-based amplification systems (TAS), nucleic acid sequence based amplification (NASBA) and 3SR (Kwoh et al., Proc. Nat. Acad. Sci. USA, 86: 1173, 1989 and PCT Application WO 88/10315).

European Application No. 329,822 discloses a nucleic acid amplification process involving cyclically synthesizing single-stranded RNA (“ssRNA”), ssDNA, and double-stranded DNA (dsDNA), which may be used in accordance with the present invention. Because of the cyclical nature of this process, the starting sequence may be chosen to be in the form of either DNA or RNA.

Miller et al., PCT Application WO 89/06700 disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR.” Frohman, (1990) and Ohara et al., (1989).

Nucleic Acid Ligand Labels

For certain embodiments, it may be appropriate to incorporate a label into nucleic acid ligands, amplification products, probes or primers. A number of different labels may be used, such as fluorophores, chromophores, radioisotopes, enzymatic tags, antibodies, chemiluminescent, electroluminescent, affinity labels, etc. One of skill in the art will recognize that these and other label moieties not mentioned herein can be used in the practice of the present invention.

Examples of affinity labels include an antibody, an antibody fragment, a receptor protein, a hormone, biotin, DNP, and any polypeptide/protein molecule that binds to an affinity label.

Examples of enzymatic tags include urease, alkaline phosphatase or peroxidase. Colorimetric indicator substrates can be employed with such enzymes to provide a detection means visible to the human eye or spectrophotometrically.

Exemplary fluorophores of use in the present invention include, but are not limited to, Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red. These and other fluorophores can be obtained from standard commercial sources (e.g., Molecular Probes, Eugene, Oreg.).

Imaging Agents and Radioisotopes

In certain embodiments, the nucleic acid ligands of the present invention may be attached to imaging agents of use for imaging, treatment and diagnosis of various diseased organs or tissues. Many appropriate imaging agents are known in the art, as are methods for their attachment to nucleic acids. Certain attachment methods involve the use of a metal chelate complex employing, for example, an organic chelating agent such a DTPA attached to the nucleic acid.

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

Radioisotopes of potential use as imaging or therapeutic agents include astatine²¹¹, ¹⁴carbon, ⁵¹chromium, ³⁶chlorine, ⁵⁷cobalt, ⁵⁸cobalt, copper⁶⁷, ¹⁵²Eu, gallium ⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵I is often being preferred for use in certain embodiments, and technicium^{99m} and indium¹¹¹ are also often preferred due to their low energy and suitability for long range detection.

Methods of Immobilization of Nucleic Acid Ligands

In various embodiments, the nucleic acid ligands of the present invention may be attached to a solid surface (“immobilized”). In a preferred embodiment, immobilization may occur by attachment of an organic semiconductor to a solid surface, such as a magnetic, glass or plastic bead, a plastic microtiter plate or a glass slide. Nucleic acid ligands may be attached to the organic semiconductor by electrostatic interaction with magnesium ion. The attachment of nucleic acid ligand may be readily reversed by addition of a magnesium chelator, such as EDTA.

Immobilization of nucleic acid ligands may alternatively be achieved by a variety of methods involving either non-covalent or covalent interactions between the immobilized nucleic acid ligand, comprising an anchorable moiety, and an anchor. In an exemplary embodiment, immobilization may be achieved by coating a solid surface with streptavidin or avidin and the subsequent attachment of a biotinylated polynucleotide (Holmstrom et al., Anal. Biochem. 209:278-283, 1993). Immobilization may also occur by coating a polystyrene or glass solid surface with poly-L-Lys, followed by covalent attachment of either amino- or sulfhydryl-modified polynucleotides, using bifunctional crosslinking reagents (Running et al., BioTechniques 8:276-277, 1990; Newton et al. Nucl. Acids Res. 21:1155-1162, 1993).

Immobilization may take place by direct covalent attachment of short, 5′-phosphorylated primers to chemically modified polystyrene plates (Rasmussen et al., Anal. Biochem, 198:138-142, 1991). The covalent bond between the modified oligonucleotide and the solid phase surface is formed by condensation with a water-soluble carbodiimide. This method facilitates a predominantly 5′-attachment of the oligonucleotides via their 5′-phosphates.

U.S. Pat. No. 5,610,287, incorporated herein by reference, discloses a method of noncovalently immobilizing nucleic acid ligand molecules in the presence of a salt or cationic detergent on a hydrophilic polystyrene solid support containing an —OH, —C═O or —COOH hydrophilic group or on a glass solid support. The support is contacted with a solution having a pH of about 6 to about 8 containing the nucleic acid ligand and the cationic detergent or salt. The support containing the immobilized nucleic acid ligand may be washed with an aqueous solution containing a non-ionic detergent without removing the attached molecules.

Another commercially available method for immobilization is the “Reacti-Bind™DNA Coating Solutions”. This product comprises a solution that is mixed with DNA and applied to surfaces such as polystyrene or polypropylene. After overnight incubation, the solution is removed, the surface washed with buffer and dried, after which it is ready for hybridization. It is envisioned that similar products, i.e. Costar “DNA-BIND™” or Immobilon-AV Affinity Membrane (IAV, Millipore, Bedford, Mass.) may be used in the practice of the instant invention.

Cross-Linkers

Bifunctional cross-linking reagents may be of use in various embodiments of the claimed invention, such as attaching an organic semiconductor to a nucleic acid ligand, attaching an organic semiconductor to a substrate, attaching various functional groups to a nucleic acid ligand, or attaching a nucleic acid ligand or an analyte to a bead or particle. Homobifunctional reagents that carry two identical functional groups are highly efficient in inducing cross-linking. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, guanidino, indole, or carboxyl specific groups. Of these, reagents directed to free amino groups have become especially popular because of their commercial availability, ease of synthesis and the mild reaction conditions under which they can be applied.

Exemplary methods for cross-linking molecules, such as organic semiconductors, nucleic acid ligands or analytes, are disclosed in U.S. Pat. Nos. 5,603,872 and 5,401,511. Various ligands can be covalently bound to surfaces through the cross-linking of amine residues. Amine residues may be introduced onto a surface through the use of aminosilane. Coating with aminosilane provides an active functional residue, a primary amine, on the surface for cross-linking purposes. Ligands are bound covalently to discrete sites on the surfaces. The surfaces may also have sites for non-covalent association. To form covalent conjugates of ligands and surfaces, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the silane-coated surface and organic semiconductors, nucleic acid ligands or analytes may be accomplished.

Separation and Quantitation of Nucleic Acid Ligands

It may be preferred to separate nucleic acid ligands of different lengths for the purpose of quantitation, analysis or purification. In one embodiment, amplification products may be separated by agarose, agarose-acrylamide or polyacrylamide gel electrophoresis using standard methods (Sambrook et al., 1989). Separation by electrophoresis is based upon the differential migration through a gel according to the size and ionic charge of the molecules in an electrical field. High resolution techniques normally use a gel support for the fluid phase. Examples of gels used are starch, acrylamide, agarose or mixtures of acrylamide and agarose. The gel may be a single concentration or a gradient in which pore size decreases with migration distance. In gel electrophoresis of polynucleotides, mobility depends primarily on molecular size. In pulse field electrophoresis, two fields are applied alternately at right angles to each other to minimize diffusion mediated spread of large linear polymers.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, Physical Biochemistry Applications to Biochemistry and Molecular Biology, 2nd ed. Wm. Freeman and Co., New York, N.Y., 1982). In yet another alternative, cDNA products labeled with biotin or antigen can be captured with beads bearing avidin or antibody, respectively.

Microfluidic techniques of use include separation on a platform such as microcapillaries (ACLARA BioSciences Inc., Mountain View, Calif.) or the LabChip™ liquid integrated circuit (Caliper Technologies Inc., Mountain View, Calif.). Microfluidic platforms require only nanoliter volumes of sample. Miniaturizing some of the processes involved in genetic analysis has been achieved using microfluidic devices. For example, published PCT Application No. WO 94/05414 reports an integrated micro-PCR™ apparatus for collection and amplification of nucleic acids from a specimen. U.S. Pat. No. 5,856,174, incorporated herein by reference, discloses an apparatus that combines the various processing and analytical operations involved in nucleic acid analysis.

In some embodiments, it may be preferred to provide an additional, or alternative means for analyzing nucleic acid ligands, such as microcapillary arrays. Microcapillary array electrophoresis generally involves the use of a thin capillary or channel that may or may not be filled with a separation medium. Electrophoresis of a sample through the capillary provides a size based separation profile for the sample. The use of microcapillary electrophoresis in size separation of nucleic acids has been reported in, e.g., Woolley and Mathies (Proc Natl Acad Sci USA, 91:11348-52, 1994). The high surface to volume ratio of these capillaries allows for the application of higher electric fields without substantial thermal variation, allowing for more rapid separations. When combined with confocal imaging methods, these methods provide sensitivity in the range of attomoles.

Microfabrication of microfluidic devices including microcapillary electrophoretic devices is known (e.g., Jacobsen et al., Anal. Chem., 66:1107-1113, 1994; Effenhauser et al., Anal. Chem., 66:2949-2953, 1994; Harrison et al., Science, 261:895-897, 1993; Effenhauser et al., Anal. Chem., 65:2637-2642, 1993; Manz et al., J. Chromatogr., 593:253-258, 1992; U.S. Pat. No. 5,904,824, incorporated herein by reference). Typically, these methods comprise photolithographic etching of micron scale channels on silica, silicon or other crystalline substrates or chips. In some embodiments, the capillary arrays may be fabricated from the same polymeric materials used for the fabrication of the body of the device, using injection molding techniques.

In many capillary electrophoresis methods, the capillaries, e.g., fused silica capillaries or channels etched, machined or molded into planar substrates, are filled with an appropriate separation/sieving matrix. Typically, a variety of sieving matrices are known in the art may be used in the microcapillary arrays. Examples of such matrices include, e.g., hydroxyethyl cellulose, polyacrylamide, agarose and the like. Exemplary running buffers may include denaturants and/or chaotropic agents such as urea or the like, to denature nucleic acid ligands in the sample.

Organic Semiconductors

DAT

In preferred embodiments, the organic semiconductor of use in the disclosed compositions, methods and apparatus is DAT (polydiazoaminotyrosine). DAT may be produced by reacting 3-amino-L-tyrosine (3AT), with an alkali metal nitrite, such as NaNO₂. In preferred embodiments, the 3AT is dissolved first in an aqueous or similar medium before reaction with NaNO₂. Surprisingly, the product of this reaction exhibits spectroscopic properties similar to DALM (U.S. Pat. No. 6,303,316). DALM is synthesized using luminol, a known luminescent compound.

Since diazotization reactions are, in general, exothermic, in some embodiments the reaction may be carried out under isothermal conditions or at a reduced temperature, such as, for example, at ice bath temperatures. The reaction may be carried out with refluxing for 1 hour, 2 hours, 4 hours, 6 hours or preferably 8 hours, although longer reaction periods of 10, 12, 14, 18, 20 or even 24 hours are contemplated.

DAT may be precipitated from aqueous solution by addition of a solvent in which DAT is not soluble, such as acetone. After centrifuging the precipitate and discarding the supernatant, the solid material may be dried under vacuum.

In general, the quantities of the 3AT and alkali metal nitrite reactants used are equimolar. It is, however, within the scope of the invention to vary the quantities of the reactants. The molar ratio of 3AT:metal nitrite may be varied over the range of about 0.6:1 to 3:1.

In alternative embodiments, DAT may be partially or fully oxidized prior to use, resulting in the production of oxidized-DAT (O-DAT). Reduced DAT is dissolved in 5 ml of distilled water with 0.2 gm of sodium bicarbonate added. Five milliliters of 30% hydrogen peroxide is added and the mixture is refluxed until the color of the solution changes from brown to yellow. The mixture is cooled, dialyzed against distilled water and lyophilized. The lyophilized powder contains O-DAT.

In certain embodiments, an organic semiconductor such as DAT may be used to neutralize various agents, including but not limited to anthrax spores (Kiel et al., Bioelectromagnetics 20:46-51,1999a; Kiel et al., Bioelectromagnetics 20:216-223, 1999b), Shiga toxin and/or Shiga-like toxin. The energy transducing properties of organic semiconductors facilitate the inactivation of agents by microwaves, visible light, ultraviolet, infrared or radiofrequency irradiation or exposure to pulsed corona radiation (Titan Industries, San Diego, Calif.). Although the precise mechanism by which organic semiconductors facilitate agent inactivation is unknown, it is possible that the organic semiconductor can absorb various types of radiation and convert it to heat, resulting in explosive heating of membrane bound agents or in thermal denaturation of non-membrane bound agents.

In alternative embodiments, nucleic acid ligands that bind to an analyte, such as Shiga toxin and/or Shiga-like toxin, with high affinity can be used to inactivate or destroy the analyte. A high affinity nucleic acid ligand may be attached to an organic semiconductor, such as DAT. The DAT/nucleic acid ligand couplet, after binding to the analyte, may be activated by a variety of techniques, including exposure to sunlight, heat, or irradiation of various types, including laser, microwave, radiofrequency, ultraviolet, pulsed corona and infrared. Activation of the DAT/nucleic acid ligand couplet results in absorption of energy, which may be transmitted to the analyte, inactivating or destroying it.

In other embodiments, organic semiconductors such as DAT may be operably coupled to one or more nucleic acid ligands and used to detect analytes. In such embodiments, binding of analyte to the organic semiconductor:nucleic acid ligand couplet may result in a change in the photochemical properties of the couplet that is detectable, for example, as a change in the light emission spectrum of the couplet.

DALM

In certain embodiments, diazoluminomelanin (DALM) may be used as an organic semiconductor. Production and use of DALM has been disclosed in U.S. Pat. Nos. 5,856,108 and 5,003,050, incorporated herein by reference. DALM is prepared by reacting 3AT (3-amino-L-tyrosine) with an alkali metal nitrite, such as sodium nitrite, and thereafter reacting the resulting diazotized product with luminol. At some point in the reaction, the alaninyl portion of the 3AT rearranges to provide the hydroxyindole portion of the final product. It is believed that such rearrangement occurs following coupling of the luminol to the diazotized 3AT.

The reaction between 3AT and the alkali metal nitrite is carried out in aqueous medium. Since diazotization reactions are, in general, exothermic, it may be preferred to carry out this reaction under isothermal conditions or at a reduced temperature, such as, for example, at ice bath temperatures. The reaction time for the diazotization can range from about 1 to 20 minutes, preferably about 5 to 10 minutes.

Because of the relative insolubility of luminol in aqueous medium, the luminol is dissolved in an aprotic solvent, such as dimethylsulfoxide (DMSO), then added with stirring to the aqueous solution of diazotized 3AT. This reaction is carried out at reduced temperature for about 20 to 200 minutes. The solvent is then removed by evaporation at low pressure, with moderate heating, e.g., about 30° to 37° C.

The reaction mixture is acidic, having a pH of about 3.5. The coupling of the luminol and the diazotized 3AT can be facilitated by adjusting the pH of the reaction mixture to about 5.0to 6.0.

The product DALM may be precipitated from the reaction mixture by combining the reaction mixture with an excess of a material that is not a solvent for the DALM, e.g., acetone. After centrifuging the precipitate and discarding the supernatant, the solid material may be dried under vacuum.

In general, the quantities of the 3AT, alkali metal nitrite and luminol reactants are equimolar. It is, however, within the scope of the invention to vary the quantities of the reactants. The molar ratio of 3AT:luminol may be varied over the range of about 0.6:1 to 3:1.

DALM is water soluble, having an apparent pKa for solubility about pH 5.0. DALM does not require a catalyst for chemiluminescence. The duration of the reaction is in excess of 52 hours. In contrast, luminol requires a catalyst. With micro peroxidase as the catalyst, luminol has shown peak luminescence at 1 sec and half-lives of light emission of 0.5 and 4.5 sec at pH 8.6 and 12.6, respectively. The chemiluminescence yield of DALM is better at pH 7.4 than at pH 9.5, although it still provides a strong signal at strongly basic pHs. DALM also produces chemiluminescence at pH 6.5 which is about the same intensity as that produced at pH 9.5.

DALM can be used for chemiluminescent immunoassays for biological and chemical agents; in radiofrequency and ionizing radiation dosimeters; and for RNA/DNA hybridization assays for viruses and genetic detection.

Radiation Sources

High Powered Pulse Microwave Irradiation

In certain embodiments, high power pulsed microwave radiation (HPM) applied to solutions containing an organic semiconductor, dissolved carbon dioxide (or bicarbonate), and hydrogen peroxide activates the organic semiconductor by generating sound, pulsed luminescence and electrical discharge. In one embodiment, an organic semiconductor, pulsed with microwave radiation, may act as a photochemical transducer, releasing an intense pulse of visible light and electrical discharge that may neutralize or destroy bioagents such as Shiga toxin and/or Shiga-like toxin. Infectious bioagents exposed to organic semiconductors and pulsed with microwave radiation experience damage comparable to short time, high temperature insults, although measured localized temperatures were insufficient to cause the observed effects.

Pulsed Corona Reactor (PCR) Apparatus

In alternative embodiments, a source of pulsed corona discharge, such as a pulsed corona reactor (PCR) (Titan Pulse Sciences Division, San Leandro, Calif.) may be used to create a non-thermal plasma source. This plasma constitutes a fourth state of matter, possessing anti-microbial activity. The anti-microbial activity of pulsed corona discharge may be enhanced by using organic semiconductors.

A PCR apparatus typically comprises two subassemblies—the control cabinet and the pulser/reactor combination. The control cabinet houses the electronic and gas controls required to regulate the high voltage charging power supply as well as the pulse power delivered to the reactor gas. The pulser/reactor assembly contains the pulse power generator and pulsed corona discharge reaction chambers. These two sub-assemblies are connected by a high voltage cable for charging the capacitors in the pulsed power system and by high-pressure gas lines for controlling the voltage delivered to the reactor. Electrical and switch gas supplies are connected to the control cabinet. The reactor gas supply and exhaust lines are connected directly to the reactor. The PCR unit may contain test ports with sample pin holders located on two reactor tubes and an exhaust manifold.

Detection Units

In certain embodiments of the invention, nucleic acid ligands tagged with a label, such as an organic semiconductor, may be detected using a light source and photodetector, such as a diode-laser illuminator and fiber-optic or phototransistor detector. (E.g., Sepaniak et al., J. Microcol. Separations 1:155-157, 1981; Foret et al., Electrophoresis 7:430-432, 1986; Horokawa et al., J. Chromatog. 463:39-49 1989; U.S. Pat. No. 5,302,272.) Other exemplary light sources include vertical cavity surface-emitting lasers, edge-emitting lasers, surface emitting lasers and quantum cavity lasers, for example a Continuum Corporation Nd-YAG pumped Ti:Sapphire tunable solid-state laser and a Lambda Physik excimer pumped dye laser. Other exemplary photodetectors include photodiodes, avalanche photodiodes, photomultiplier tubes, multianode photomultiplier tubes, phototransistors, vacuum photodiodes, silicon photodiodes, and charge-coupled devices (CCDs). The label, such as an organic semiconductor, may be excited to a higher energy state by the use of a light source. Return to a lower energy state is accompanied by emission of light, normally at a longer wavelength, which may be detected using a photodetector.

In certain embodiments of the invention, the detector may be positioned perpendicular to the light source to minimize background light. The photons generated by excitation of the label on the nucleic acid ligand may be collected, for example, by a fiber optic. The collected photons are transferred to a CCD detector and the light detected and quantified. In some embodiments of the invention, an avalanche photodiode (APD) may be used to detect low light levels. The APD process uses photodiode arrays for electron multiplication effects (U.S. Pat. No. 6,197,503). Alternative examples of photodetectors are known in the art (e.g., U.S. Pat. No. 5,143,8545) and any known detector and/or light source may be used.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of High Affinity Nucleic Acid Ligands Against Shiga-Like Toxin I

In vitro selection of nucleic acid ligands was initiated with a population of synthetic ssDNA that contained a region of randomized sequences (40-mers) flanked by fixed sequences (25- and 21-mers) that served as primer binding sites. The pool of nucleic acid ligands was exposed to purified Shiga-like toxin I and ligands binding to the toxin were separated from non-binding ligands. After multiple rounds of selection and amplification the highly selected nucleic acid ligands were cloned and sequenced (SEQ ID NO:1 to SEQ ID NO:11).

Materials

Purified Shiga-like toxin I with subunits A and B was purchased from Calbiochem (La Jolla, Calif.). Oligonucleotides for the nucleic acid ligand pool and amplification primers were purchased from Genosys (The Woodlands, Tex.). Taq polymerase was obtained from Display Systems Biotech (Vista, Calif.). The dNTP mix was from Applied Biosystems (Foster City, Calif.). Ultra pure urea, acrylamide/bis, fluor-coated TLC plates and buffer saturated phenol were from Ambion (Austin Tex.). Glycogen and streptavidin beads were from Roche Molecular Biochemicals (Indianapolis, Ind.). Spin columns and 10×TBE buffer from BioRad (Hercules, Calif.). Nitrocellulose discs were purchased from Millipore (Bedford, Mass.). All other reagent grade chemicals were from Sigma/Aldrich (St. Louis, Mo.).

Nucleic Acid Ligand Pool and Primers

The starting nucleic acid ligand pool was composed of 86-mers, containing 40-mer random DNA sequences (N40) attached to 5′ and 3′ fixed primer annealing sequences, as shown in Table 3 below.

TABLE 3

| 5′ Fixed sequences for primer | | 3′ Fixed sequences for |
|---|---|---|
| annealing | Random sequences | primer annealing |
| 5′-CCCCTGCAGGTGATTTT | NNNN---NNNN (40N) | 5′-AGTATCGCTAATCA |
| GCTCAAGT-3′ | | GGCGGAT-3′ |
| (SEQ ID NO:12) | | (SEQ ID NO:13) |

In Table 3, N represents an equal mixture of all four nucleotides (A, G, T and C). The 5′ end of the 5′ fixed sequence was covalently attached to three biotin residues to facilitate binding of the nucleic acid ligands to streptavidin.

The primer sequences were used to amplify the nucleic acid ligands were as shown below.

| | |
|---|---|
| 5′ Primer (25-mer) | |
| 5′-CCCCTGCAGGTGATTTTGCTCAAGT-3′ | (SEQ ID NO:12) |
| 3′ Primer (21-mer) | |
| 5′-ATCCGCCTGATTAGCGATACT-3′ | (SEQ ID NO:14) |

The 5′ end of the 5′ primer was covalently attached to three biotin residues.

PCR Amplification

The nucleic acid ligand pool was PCR amplified using equal concentrations of the 5′ and 3′ primers indicated above. PCR conditions were checked using a 200 μL reaction with 5 pmol of template and 0.1 μM of each primer, 20 μL of 10× PCR reaction buffer supplied by the manufacturer, 4 μL of 10 mM dNTP mix and 5 units of display TAQ polymerase, with sterile distilled water added to 200 μL. Optimal PCR conditions were determined to be denaturation at 94° C. for 3 minutes, annealing at 45° C. for 30 seconds, primer extension at 72° C. for 1 minute and a final extension at 72° C. for 3 minutes. The reaction was performed using a Stratagene Corp. (La Jolla, Calif.) RoboCyclerO Model 96 thermal cycler with a “Hot Top” assembly. The contents of reaction were checked for every 3^{rd} cycle and the optimal number of cycles was determined. After obtaining all the required optimal conditions the original nucleic acid ligand pool was amplified using a master mix of 25 mL reaction. The amplified DNA pool was recovered by ethanol precipitation in the presence of glycogen and the final DNA pellet was resuspended in sterile TE buffer [Tris-HCl, EDTA, pH 8.0] and used for streptavidin binding.

Streptavidin Binding and Elution of ssDNA

PCR amplified double stranded DNA was mixed with streptavidin-agarose beads and incubated at room temperature for one hour to bind biotin-labeled ssDNA to the beads. The mixture was transferred to spin columns and denatured by addition of 0.2 M NaOH. The biotin labeled DNA strand remained in the column along with the streptavidin beads, while the unlabeled strand passed through the column and was collected. The eluate containing unlabeled ssDNA was neutralized with 3 M sodium acetate (pH 5.0) and ethanol precipitated overnight and recovered by centrifugation at 4° C. at 13,000 rpms. The precipitated ssDNA was further purified by gel electrophoresis.

Gel Purification of ssDNA

The ssDNA was mixed with denaturing 2× sample buffer containing 90% formamide, 1 mM EDTA and 0.1 percent bromophenol blue and heated at 90° C. for 5 minutes. After cooling to room temperature the contents were separated using 9% acrylamide and 7 M urea with 1× TBE buffer as the running buffer. ssDNA was visualized under short wave length UV light. The appropriate band containing nucleic acid ligands was cut out and eluted overnight in 0.3 M sodium chloride, then ethanol precipitated at −80° C. Following centrifugation at 4° C. for 30 minutes, ssDNA was collected and used for further analysis.

In Vitro Selection

To exclude filter-binding ssDNA sequences from the pool, the DNA was passed over a 0.45 μm HAWP filter (Millipore, Bedford, Mass.) and washed with an equal volume of binding buffer. The filtrate containing unbound ssDNA was used for in vitro selection. In general the final yield of ssDNA was in micromolar range. One hundred pmol of ssDNA (nucleic acid ligands) was incubated with 100 pmol of recombinant holo-protein Shiga-like toxin I (Calbiochem, Calif.) in a binding buffer containing 20 mM Tris-HCl, pH 7.25, 45 mM sodium chloride, 3 mM magnesium chloride, 1 mM EDTA and 1 mM dithioerythritol in a final volume of 250 μL. The binding reaction mixture was incubated for one hour at room temperature. After binding, the solution was vacuum filtered over a HAWP filter at 5 p.s.i. and washed five times (5×0.2 ml ) with binding buffer. ssDNA that bound to Shiga-like toxin I protein was retained on the filter. Retained ssDNA was eluted twice with 0.2 ml of 7 M urea, 100 mM MES (4-Morpholine-ethanesulfonic acid, Roche Molecular Biochemicals, Indianapolis, Ind.) pH 5.5 and 3 mM EDTA for 3 min at 100° C.

The eluted ssDNA, comprising nucleic acid ligands with an affinity for Shiga-like toxin I, was extracted once with phenol: chloroform and precipitated overnight with an equal volume of isopropanol in the presence of glycogen. The ssDNA was recovered after centrifugation at 4° C. and used for next round of amplification. The stringency of selection was increased by negative selection after each cycle. After three rounds of selection and amplification, dsDNA molecules were cloned into the pCR II-TOPO vector (Invitrogen, Austin, Tex.). The selected clones were sequenced by standard techniques. The sequences of Shiga-like toxin I binding nucleic acid ligands are disclosed below in SEQ ID NO:1 through SEQ ID NO:11.

SEQ ID NO:1

5′-CAGCCCCTTCTCCCCCTGACCCTATATCTTCATCTACCGT-3′

SEQ ID NO:2

5′-GCCACTCTCTAAATACTGACCCGACCTAACTGTTTGATAT-3′

SEQ ID NO:3

5′-GTACTACCACCCACCCAGCCTCATCCTACAAATTCTATCC-3′

SEQ ID NO:4

5′-GCCCCCTCCTTACCTAGCCCACCCGCTCGTTATACCTTCC-3′

SEQ ID NO:5

5′-GCGCGCCGCTCTTATTCGACACTGTTTGGCCCTTATTGAT-3′

SEQ ID NO:6

5′-GCGCAGCCATCCCCTTGTACATATCTAACCTTTTCTCCA-3′

SEQ ID NO:7

5′-GCACCCAACATCATCCTCATATTTCATTATACTTACGTCT-3′

SEQ ID NO:8

5′-GGTAACTAGCATTCATTTCCCACACCCGACCCGTCCATAT-3′

SEQ ID NO:9

5′-CCCCCCTCCTACACAACGCCCAGCAATTGTAATTCGTCCC-3′

SEQ ID NO:1O

5′-GGCAACCCTAACCATCAAACCCGCACTTAATCCAATATTC-3′

SEQ ID NO:11

5′-CCCACTCCCCATCCACGCTCACCCCTTTGGCAATTCCTCA-3′

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for preparing one or more nucleic acid ligands against Shiga toxin and/or Shiga-like toxin comprising:
a) obtaining a pool of nucleic acid ligands;
c) contacting the nucleic acid ligands with Shiga toxin and/or Shiga-like toxin;
d) separating ligands bound to Shiga toxin and/or Shiga-like toxin from ligands that do not bind to Shiga toxin and/or Shiga-like toxin; and
e) obtaining one or more nucleic acid ligands that bind to Shiga toxin and/or Shiga-like toxin.

2. The method of claim 1, further comprising repeating (c) and (d) until one or more nucleic acid ligands of a selected degree of specificity and/or binding affinity against Shiga toxin and/or Shiga-like toxin is obtained.

3. The method of claim 2, wherein the nucleic acid ligand binds to Shiga toxin and/or Shiga-like toxin with high affinity.

4. The method of claim 2, wherein the nucleic acid ligand is highly specific for Shiga toxin and/or Shiga-like toxin.

5. The method of claim 4, wherein the nucleic acid ligand binds only to Shiga toxin and/or Shiga-like toxin.

6. The method of claim 1, wherein the pool of nucleic acid ligands is attached to magnetic beads.

7. The method of claim 1, wherein the nucleic acid ligands are operably linked to an organic semiconductor.

8. The method of claim 7, wherein the organic semiconductor is diazotyrosine (DAT) or diazoluminomelanin (DALM).

9. The method of claim 1, wherein said separating comprises nitrocellulose filtration.

10. The method of claim 9, wherein nucleic acid ligands that bind to nitrocellulose filters in the absence of Shiga toxin and/or Shiga-like toxin are removed from the pool of nucleic acid ligands before contacting the Shiga toxin and/or Shiga-like toxin with the pool.

11. The method of claim 1, wherein the nucleic acid ligands comprise 40-mers of random sequence, the random 40-mers attached at their 5′ and 3′ ends to primer binding sequences.

12. The method of claim 11, further comprising amplifying the nucleic acid ligands using a 5′ primer and a 3′ primer.

13. The method of claim 11, wherein one of the primers is labeled with biotin.

14. The method of claim 13, wherein the pool of nucleic acid ligands comprises single-stranded DNA (ssDNA) prepared by binding biotin-labeled nucleic acid ligands to streptavidin conjugated beads.

15. A composition comprising one or more nucleic acid ligands that bind to Shiga toxin and/or Shiga-like toxin.

16. The composition of claim 15, wherein the one or more ligands comprise at least six contiguous nucleotides having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 and SEQ ID NO:11.

17. The composition of claim 16, wherein the one or more ligands comprise at least 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 contiguous nucleotides having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 and SEQ ID NO:11.

18. The composition of claim 17, wherein the one or more ligands comprise a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 and SEQ ID NO:11.

19. The composition of claim 16, wherein said nucleic acid ligand is incorporated into a vector.

20. The composition of claim 16, further comprising an organic semiconductor attached to said nucleic acid ligand.

21. A nucleic acid ligand prepared by the method of claim 1 or claim 2.

22. The nucleic acid ligand of claim 21, wherein the nucleic acid ligand is attached to an organic semiconductor.

23. The nucleic acid ligand of claim 21, wherein the nucleic acid ligand is incorporated into a vector.

24. A method of neutralizing Shiga toxin and/or Shiga-like toxin comprising:
a) preparing at least one nucleic acid ligand that binds to Shiga toxin and/or Shiga-like toxin;
b) attaching the nucleic acid ligand to an organic semiconductor;
c) exposing Shiga toxin and/or Shiga-like toxin to the nucleic acid ligand and organic semiconductor;
d) activating the organic semiconductor; and
e) neutralizing Shiga toxin and/or Shiga-like toxin.

25. The method of claim 24, wherein said activating comprises exposing the organic semiconductor to sunlight, heat, laser radiation, ultraviolet radiation, infrared radiation, radiofrequency radiation, microwave radiation or pulse corona discharge.

26. The method of claim 24, wherein said nucleic acid ligand comprises at least six contiguous nucleotides having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 and SEQ ID NO:11.

27. A method of detecting Shiga toxin and/or Shiga-like toxin comprising:
a) obtaining at least one nucleic acid ligand that binds to Shiga toxin and/or Shiga-like toxin;
b) exposing a sample to the nucleic acid ligand; and
c) detecting Shiga toxin and/or Shiga-like toxin bound to the nucleic acid ligand.

28. The method of claim 27, wherein the nucleic acid ligand is labeled.

29. The method of claim 28, wherein the nucleic acid ligand is labeled with an organic semiconductor.

30. The method of claim 27, wherein the nucleic acid ligand comprises at least six contiguous nucleotides having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 and SEQ ID NO:11.

31. The method of claim 27, wherein the nucleic acid ligands are attached to magnetic beads.

32. The method of claim 31, further comprising distributing the magnetic beads in an environment suspected of containing Shiga toxin and/or Shiga-like toxin.

33. The method of claim 32, further comprising collecting the magnetic beads from the environment and testing them for attached Shiga toxin and/or Shiga-like toxin.


SEQUENCE LISTING (14 sequences)

SEQ ID NO: 1 - DNA, 40 bp, Artificial
  Synthetic Oligonucleotide
cagccccttctccccctgaccctatatcttcatctaccgt

SEQ ID NO: 2 - DNA, 40 bp, Artificial
  Synthetic Oligonucleotide
gccactctctaaatactgacccgacctaactgtttgatat

SEQ ID NO: 3 - DNA, 40 bp, Artificial
  Synthetic Oligonucleotide
gtactaccacccacccagcctcatcctacaaattctatcc

SEQ ID NO: 4 - DNA, 40 bp, Artificial
  Synthetic Oligonucleotide
gccccctccttacctagcccacccgctcgttataccttcc

SEQ ID NO: 5 - DNA, 40 bp, Artificial
  Synthetic Oligonucleotide
gcgcgccgctcttattcgacactgtttggcccttattgat

SEQ ID NO: 6 - DNA, 39 bp, Artificial
  Synthetic Oligonucleotide
gcgcagccatccccttgtacatatctaaccttttctcca

SEQ ID NO: 7 - DNA, 40 bp, Artificial
  Synthetic Oligonucleotide
gcacccaacatcatcctcatatttcattatacttacgtct

SEQ ID NO: 8 - DNA, 40 bp, Artificial
  Synthetic Oligonucleotide
ggtaactagcattcatttcccacacccgacccgtccatat

SEQ ID NO: 9 - DNA, 40 bp, Artificial
  Synthetic Oligonucleotide
cccccctcctacacaacgcccagcaattgtaattcgtccc

SEQ ID NO: 10 - DNA, 40 bp, Artificial
  Synthetic Oligonucleotide
ggcaaccctaaccatcaaacccgcacttaatccaatattc

SEQ ID NO: 11 - DNA, 40 bp, Artificial
  Synthetic Oligonucleotide
cccactccccatccacgctcacccctttggcaattcctca

SEQ ID NO: 12 - DNA, 25 bp, Artificial
  Synthetic Oligonucleotide
cccctgcaggtgattttgctcaagt

SEQ ID NO: 13 - DNA, 21 bp, Artificial
  Synthetic Oligonucleotide
agtatcgctaatcaggcggat

SEQ ID NO: 14 - DNA, 21 bp, Artificial
  Synthetic Oligonucleotide
atccgcctgattagcgatact